(19) United States
(12) Patent Application Publication
(10) Pub. No.: US 2011/0112558 A1
(43) Pub. Date: May 12, 2011

(54) TISSUE FIXATION SYSTEM WITH SINGLE COMPONENT ANCHOR

(75) Inventors: James Whayne, Chapel Hill, NC (US); Sidney Fleischman, Durham, NC (US); Kevin Ohashi, Jamaica Plain, MA (US); Dennis Godfrey, Marblehead, MA (US); Paul V. Fenton, Marblehead, MA (US)

(73) Assignee: OC2, LLC, a Massachusetts limited liability company, Marblehead, MA (US)

(21) Appl. No.: 12/897,383

(22) Filed: Oct. 4, 2010

Related U.S. Application Data
(60) Provisional application No. 61/248,131, filed on Oct. 2, 2009.

(51) Int. Cl. A61B 17/08

(52) U.S. Cl. 606/151

(57) ABSTRACT

A method and apparatus for biceps tenodesis or attachment of other tendon, or other soft tissue to bone. The tissue fixation system incorporates a single component anchor fabricated from a unitary piece or thin wafers bonded into a single component. The anchors incorporate features to engage a tendon or other soft tissue and maintain that engagement as the anchor and tendon are positioned into a bone tunnel or channel. The anchor secures to bone ensuring the tendon or other soft tissue are engaged within the bone tunnel or channel to produce the required fixation.

### RELATED APPLICATIONS

The present application claims the benefit of U.S. Provisional Application 61/248,131 filed Oct. 2, 2009, entitled Single Component Tenodesis Anchor System, which is hereby incorporated by reference.

### FIELD OF THE INVENTION

The present disclosure relates generally to orthopedic medical devices for directly fixing biceps tendons, other tendons, or other soft tissue, to bone. More specifically, the disclosure relates to single component anchor and their associated deployment systems that once deployed and secured into bone, attach a tendon or other soft tissue directly into a bone tunnel or channel. The dimensions of the anchors are tailored for orthopedic access with standard arthroscopic equipment. The anchors can be used in either open or arthroscopic procedures. The anchors are available in different sizes, which allow for the fixation of biceps tendon, other tendon, or other soft tissue of varying sizes and for a variety of surgical applications.

### BACKGROUND OF THE INVENTION

One of the most common needs in orthopedic surgery is the fixation of the biceps tendon or other tendon against bone. The fixation of tendon torn from its insertion site, diseased tendon, tendon torn from its attachment points or other tendons or soft tissue into a modified position commonly requires engagement of a bone anchor with the tendon and placement of the tendon and bone anchor as a combination into a hole drilled into a bone to secure the tendon, or other soft tissue within the bone tunnel or channel. Besides biceps tendon, rotator cuff and torn flexor tendons in the hand are common applications that require the use of bone anchors. Tendons are also frequently used in the reconstruction of unstable joints. Common examples include anterior cruciate ligament and collateral ligament reconstructions of the knee, medial and lateral elbow collateral ligament reconstructions, ankle collateral ligament reconstruction, and finger and hand collateral ligament reconstructions.

Traditional techniques that are used to fix tendon to bone include the use of pull-out sutures, bone tunnels, and interference screw fixation. The most common method of fixation of tendon to bone is the use of bone tunnels with either suture fixation, or interference screw fixation. Holes are drilled in the bone at right angles to the surface of the bone. After creation of the holes, discrete anchors are passed and secured into the holes. Sutures inserted through the rotator cuff, tendon, or other soft tissue are tied to the anchors.

Alternatively, an interference fit between a screw anchor and the tendon is used to secure the tendon or other soft tissue to the bone tunnel or channel. These conventional anchors require multiple pieces that move and/or rotate relative to each other at joints or require screwing into bone along the tendon or other soft tissue which may abrade, tear, or alter the orientation of the tendon within the bone tunnel or channel.

### BRIEF SUMMARY OF THE INVENTION

The present disclosure describes a system capable of securing a tendon, or other soft tissue within a bone tunnel or channel. The tissue fixation system embodiments enable engagement of the tendon with the anchor to facilitate grasping and moving the tendon and anchor combination into a bone tunnel or channel, where the anchor is tapped into place to secure the tendon or other soft tissue, without having to screw or rotate the anchor.

The present tissue fixation system incorporates a single component anchor (or stacked assembly of wafers that form a single component anchor) with no pivoting joints. In one embodiment, the single component anchor incorporates a central tissue penetrating member that engages the tendon and enables positioning the tendon into a bone tunnel or channel. Integrated arms with lateral structures, such as for example spikes or barbs, extend from the central tissue penetrating member to form an opening to partially engage and support the tendon during placement and attachment into the bone tunnel or channel. The integrated arms of the single component anchor are preferably deflected and compressed into a smaller profile to allow placement into the bone tunnel or channel and to then expand once positioned to initiate and maintain attachment to the cortical or cancellous bone. This engagement creates fixation between the biceps tendon, other tendon, or other soft tissue and the bone into which the tendon or soft tissue is inserted and anchor is tapped into engagement.

The various embodiments of the present disclosure provide a variety of single component anchors that engage tendon or other soft tissues to be repositioned into a bone tunnel or channel and allow fixation of the tendon or other soft tissue to bone without having to pass suture, move multiple parts of an anchor about joints to engage the tendon and/or bone, or rotate a screw adjacent to the tendon. Many previous bone anchors have either been screws, which require rotating the anchor adjacent to the tendon and may twist and/or abrade the tendon. Various tacks have also been used as bone anchors, which allow the pinning of adjacent tissues to bone. Also used are suture anchors, which attach a suture to bone and requires passing of the suture through the soft tissue in order to attach soft tissue to bone.

The single component anchors of the present disclosure provide engagement of the anchor to tendon or other soft tissue to allow repositioning of the tendon into the bone tunnel or channel, and reliable attachment approaches that use a single component fabricated by a single piece of material or multiple wafers bonded into a single piece. The present anchors do not have joints or parts that slide or pivot relative to each other to directly secure the tendon or other soft tissue into the bone tunnel or channel.

The present tissue fixation system includes a uniquely shaped, single component anchor that can be supported by a single instrument to engage a tendon or other soft tissue, deploy the tendon into a bone tunnel or channel and secure the tendon to bone. These anchors incorporate features that allow engagement of the anchor to tendon, provide attachment of the tendon to the anchor and secure the anchor and tendon combination within a bone tunnel or channel. The various embodiments incorporate a deployment system that permits manipulation of the anchor and tendon combination and facilitate tapping the anchor and tendon into a bone tunnel or channel to ensure fixation of the anchor thus the tendon to bone. The single component anchor incorporates features on the device to provide for removal or readjustment of the anchor from the bone tunnel.

One embodiment is directed to a tissue fixation system for attaching tissue to a channel formed in a bone. The tissue fixation system includes a generally planar, single component anchor with a partially enclosed tissue engaging region having an opening oriented in a distal direction (away from the user). The tissue engaging region is preferably adapted to compressively engage the tissue. A pair of arms extending in a generally proximal direction include structures adapted to engage with the bone. Displacement of the arms toward each other in a compressed configuration increases the size of the opening to facilitate engagement with the tissue. At least one tissue penetrating member is engaged with the anchor and extends into the tissue engaging region.

The tissue penetrating member can be integrally formed with the anchor or can be a discrete component sized to slide through the hole in the anchor and into the tissue engaging region. One or more eyelets are optionally formed in one or more of the arms. The tissue penetrating member can be inserted through the bottom of the channel, penetrating the bone as well as the tendon. The anchor optionally includes one or more serrations or barbs oriented toward the tissue engaging region. The structures on the arms are preferably configured to engage into cancellous bone and apply tension upward from under the cortical bone layer within the channel.

A deployment system is preferably provided that engages the anchor at proximal ends of the arms. The deployment system preferably maintains the arms in a compressed configuration, and releases the arms when the anchor is in the channel. In one embodiment, the deployment system includes a sheath that slidingly engages proximal ends of the arms in the compressed configuration. The distal end of the tissue penetrating member optionally includes one or more of points, blades, teeth, or serrations.

In one embodiment, the anchor is a plurality of generally planar, single component anchors laminated to form a unitary structure. One or more secondary components are provided for insertion into the channel with the anchor.

The present disclosure is also directed to a method of attaching tissue to a channel formed in a bone. The method includes the steps of compressing proximally extending arms on a generally planar, single component anchor to increase the size of a distally oriented opening to a tissue engaging region. The tissue engaging region is then engaged with the tissue. At least one tissue penetrating member extends into the tissue engaging region to engage with the tissue. The anchor and the tissue are inserted into the channel formed in the bone. The arms are released so that structures on the arms engage cortical or cancellous bone within the channel.

The step of inserting the anchor into the channel is preferably performed without rotation. The tissue penetrating member can engage with the tissue substantially simultaneously with the tissue engaging region. Alternatively, the tissue penetrating member is subsequently slid into a hole extending through the anchor and into the tissue engaging region.

The deployment system engages proximal ends of the arms. In one embodiment, the deployment system retains the arms in a compressed configuration and releases the arms when the anchor is in the channel. One or more secondary components can be inserted into the channel with the anchor.

In one embodiment, the anchor system includes one or more stay sutures that assist in locking the anchor to the deployment system. The sutures also serves as a retrieval mechanism for repositioning or removing the anchor.

The spring force generated by the arms can be engineered for different applications, such as, for example, the density of the bone in which the anchor is being deployed. A further embodiment is a design for field adjustment in situ of the expanding spring force by the surgeon user.

The method optionally includes engaging a tool with the proximal extending arms and compressing the proximally extending arms to release the structures on the arms from the cortical or cancellous bone within the channel. The anchor is then removed from the channel.

### BRIEF DESCRIPTION OF THE SEVERAL VIEWS OF THE DRAWING

FIGS. 1A to 1C are various views of a single component anchor in accordance with an embodiment of the present disclosure.

FIGS. 2A to 2E are various views of the single component anchor of FIGS. 1A to 1C engaging a tendon for deployment in accordance with an embodiment of the present disclosure.

FIGS. 3A to 3C are various views of a deployment system for a single component anchor in accordance with an embodiment of the present disclosure.

FIGS. 4A to 4C are various views of the deployment system of FIGS. 3A to 3C engaging a tendon for deployment in accordance with an embodiment of the present disclosure.

FIGS. 5A to 5D are various views of a single component anchor deployed attached to a tendon within a pre-drilled bone channel in accordance with an embodiment of the present disclosure.

FIGS. 6A to 6D are various views of a deployed single component anchor securing a tendon within a bone channel in accordance with an embodiment of the present disclosure.

FIGS. 7A to 7C are various views of an alternative deployment system for a single component anchor that maintains the anchor in a compressed during deployment in accordance with an embodiment of the present disclosure.

FIGS. 8A to 8C are various views of the deployment system in FIGS. 7A to 7C with the outer sheath retracted to allow the single component anchor to expand during deployment.

FIGS. 9A to 9E are various views of an alternate single component anchor in accordance with an embodiment of the present disclosure.

FIGS. 10A to 10C are various views of a plurality of the anchors of FIGS. 9D and 9E stacked together in a multi-layered structure in accordance with an embodiment of the present disclosure.

FIGS. 11A to 11D are various views of the anchor in FIGS. 10A to 10C connected to a deployment system in accordance with an embodiment of the present disclosure.

FIG. 12A illustrates an anchor with a removable tissue penetrating member in accordance with an embodiment of the present disclosure.

FIGS. 12B-12D schematically illustrate the operation of the anchor of FIG. 12A.

FIGS. 13A to 13C illustrate various structures for distal end of a central tissue penetrating member on an anchor in accordance with an embodiment of the present disclosure.

FIG. 14 illustrates an anchor with secondary components in accordance with an embodiment of the present disclosure.

FIGS. 15A-15C illustrate an anchor with a triangular prismatic central tissue penetrating member in accordance with an embodiment of the present disclosure.

FIGS. 16A-16C illustrate an anchor with a blade-like central tissue penetrating member with an angled sharpened tip in accordance with an embodiment of the present disclosure.

FIGS. 17A-17C illustrate an anchor with serrated gripping mechanisms in the tissue engaging region in accordance with an embodiment of the present disclosure.

FIGS. 18A-18C illustrate an alternate anchor with thinner walls in accordance with an embodiment of the present disclosure.

FIGS. 19A-19C illustrate an anchor with an elongated central tissue penetrating member that extends beyond the tissue engaging region in accordance with an embodiment of the present disclosure.

FIGS. 20A-20C illustrate an alternate anchor with an elongated central tissue penetrating member in accordance with an embodiment of the present disclosure.

FIGS. 21A-21C illustrate an anchor with notched arms that engage with a retrieving device in accordance with an embodiment of the present disclosure.

FIG. 22A-22C illustrate various views of a deployment/retrieving device in accordance with an embodiment of the present disclosure.

FIG. 23 illustrates use of suture material in combination with a single component anchor in accordance with an embodiment of the present disclosure.

### DETAILED DESCRIPTION OF DISCLOSURE

The present disclosure relates to methods and devices that enable engagement, repositioning, and direct fixation of tendons, and/or soft tissues to bone for the repair of torn or diseased tendons, or the reconstruction of unstable joints. The device and system embodiments are applicable to all surgical procedures that require direct fixation of tendon or other soft tissue to bone, such as for example, the shoulder, elbow, wrist, hand, knee, ankle, and foot.

The following is a detailed description of certain exemplary embodiments of the disclosure. This detailed description is not to be taken in a limiting sense, but is made merely for the purpose of illustrating certain general principles of the disclosure. Several exemplary embodiments of the present disclosure, and many features and advantages of those exemplary embodiments will be elaborated in the following detailed description and accompanying drawings.

### Tissue Fixation System Embodiments

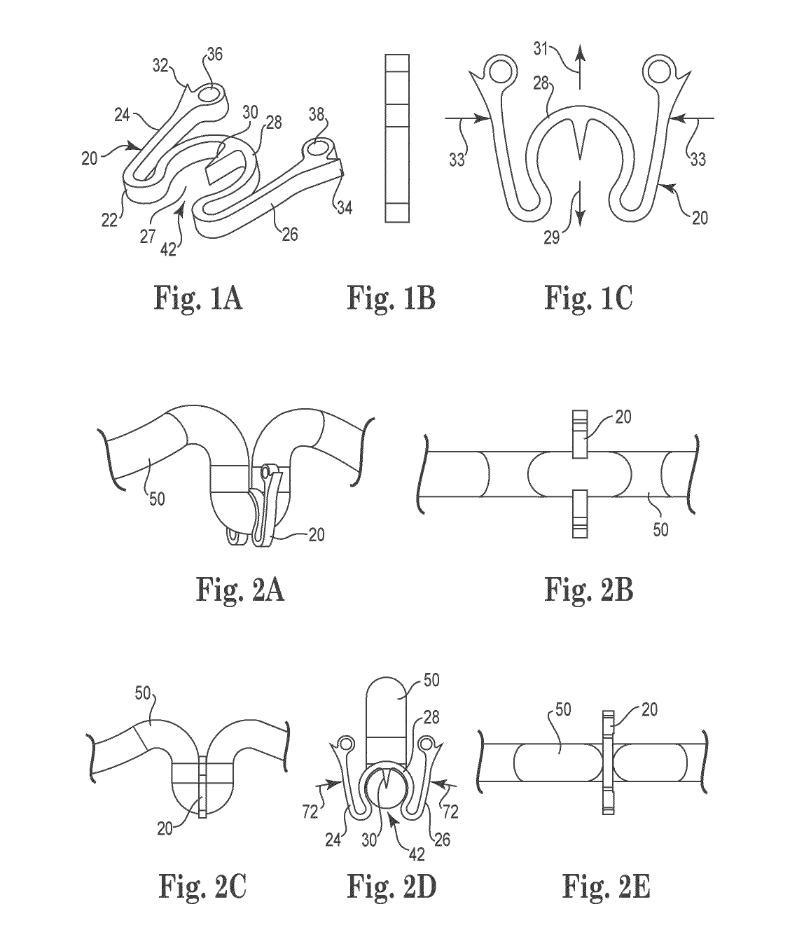

FIGS. 1A to 1C are various views of a single component anchor 20 in accordance with an embodiment of the present disclosure. The anchor 20 incorporates a unitary member 22 that is fabricated into two opposing arms 24, 26 feeding into a central curved tendon housing 28 that connects at a central tissue penetrating member 30 that facilitates partial penetration into a tendon or other soft tissue for engaging and manipulating the tendon or other soft tissue (see e.g., FIG. 2D). As used herein, “single component anchor” refers to a unitary or monolithic structure, without mechanical pivot joints, mechanical hinges, or the like. The unitary structure can be homogenous or heterogeneous, such as for example, a multi-layered stacked assembly.

The tendon housing 28 forms a partially enclosed tissue engaging region 27 with opening 42 to facilitate engagement with a tendon or other soft tissue. The opening 42 is oriented in a distal direction 29, while the arms 24, 26 are generally oriented in a proximal direction.

The opposing arms 24, 26 contain one or more structures, such as for example spikes 32, 34, that extend laterally outward to enable engaging bone within the bone tunnel and channel. Two eyelets 36, 38 are formed from the arms 24, 26 adjacent the laterally extending spikes 32, 34 to permit engaging the arms 24, 26 for deflection or other manipulation using a deployment system, as will be discussed further below. The eyelets 36, 38 can also be used to attach suture material, either to secure the tissue to the anchor 20, to secure the anchor 20 to the deployment tool, and/or to aid in removing the anchor 20. In one embodiment, suture material is configured to enhance engagement of the spikes 32, 34 with the bone.

The unitary structure of the anchor 20 permits the size and shape of the tissue engaging region 27 and the opening 42 to be manipulated by flexing the arms 24, 26 toward each other in the direction of compressive force 33. In particular, displacing the arms 24, 26 toward each other in the direction of compressive force 33 creates a compressed configuration that increases the size of the opening 42 to facilitate engagement with the tissue. When the compressive force 33 is removed, the resilience of the anchor 20 causes the arms 24, 26 and the opening 42 to resume a substantially expanded configuration. In the preferred embodiment, the tissue engaging region 27 compressively engages the tissue when the anchor 20 is in the substantially expanded configuration.

The unitary anchor 20 of this embodiment and alternative embodiments may be fabricated by extruding a rod and EDM or machining the anchor front view shape (or other shape for alternative embodiments) into the rod and cutting the anchors using EDM, laser cutting, or other mechanism to define the width of the anchor. The wall thickness 40 of the anchor 20, as defined by the side view in FIG. 1B, may be tailored to the application. For example, to connect a biceps tendon into an 8 mm bone tunnel or channel, the wall thickness preferably ranges from about 0.040″ to about 0.120″ to supply space for the tendon or other soft tissue to bend under the tendon housing 28 of the anchor 20 within the bone tunnel or channel with each end of the tendon or other soft tissue extending beyond the opening to the bone tunnel or channel. The anchor 20 may be configured to tightly compress the tendon or other soft tissue against the bone tunnel or channel, or allow space to facilitate free insertion of the tendon or other soft tissue into the bone tunnel or channel.

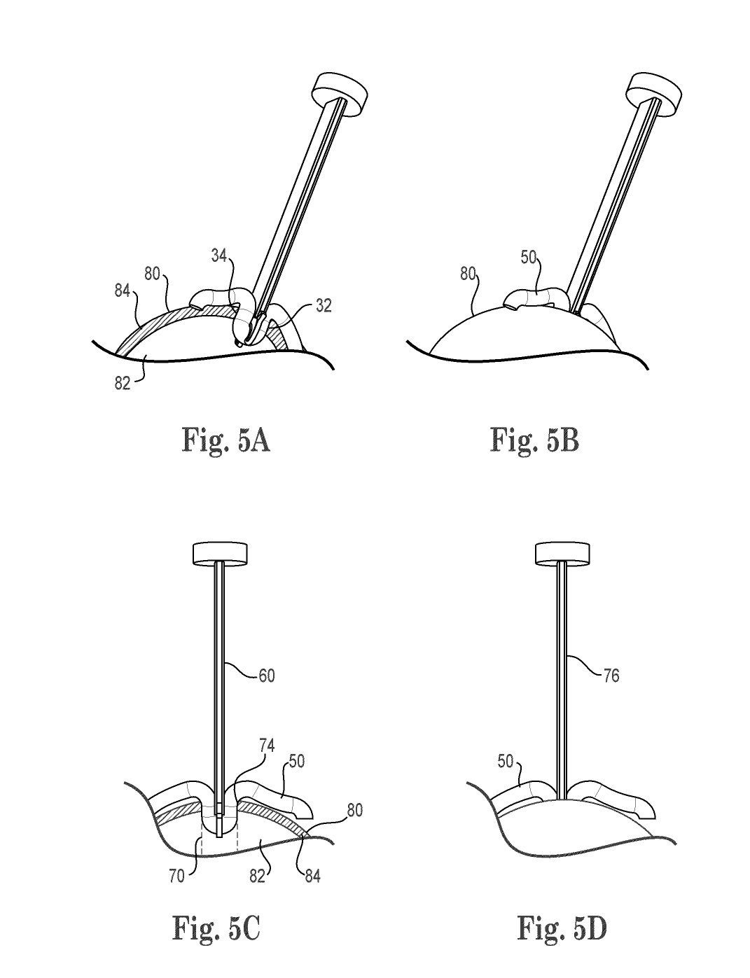

FIGS. 2A to 2E show various views of the anchor 20 of FIGS. 1A to 1C engaging a tendon or other soft tissue 50 to enable securing and manipulating of the tendon or other soft tissue 50 into the bone tunnel or channel (see e.g., FIG. 5C).

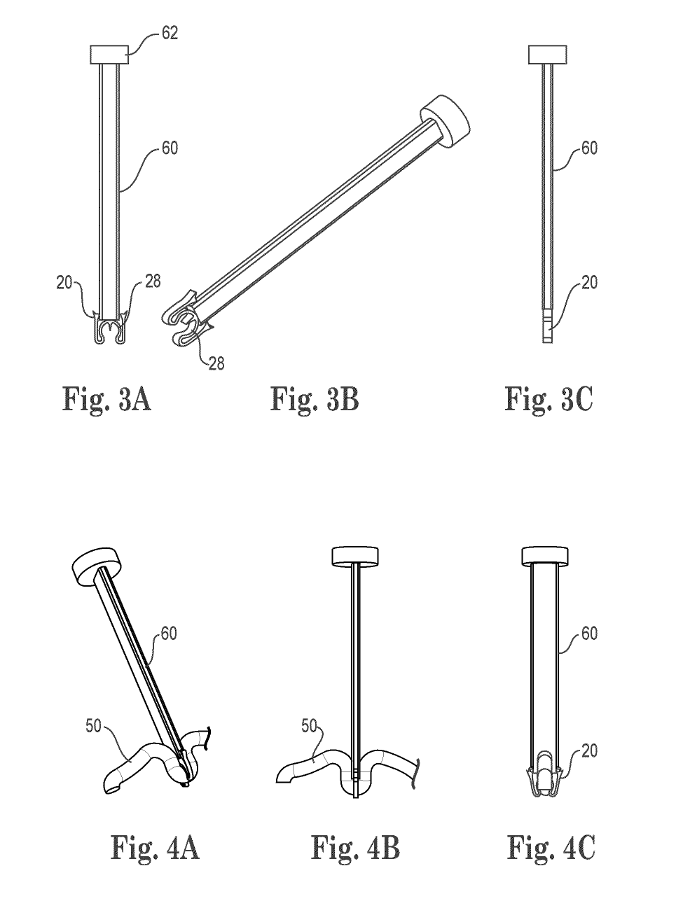

The arms 24, 26 may be compressed by squeezing them together in direction 72 manually or with features of a deployment system 60 (see FIG. 3A) that urges the arms 24, 26 together thereby flexing the curved tendon housing 28 into a larger diameter and increasing the opening 42 defined by the tendon housing 28. The central tissue penetrating member 30 may partially or completely penetrate through or into the tendon or other soft tissue 50 and possibly into the bone. This engagement ensures attachment between the anchor 20 and the tendon or other soft tissue 50. Once engaged, the arms 24, 26 of the anchor 20 are preferably expanded allowing the tendon housing 28 to return to its smaller preformed shape further engaging the tendon or other soft tissue 50. Alternatively, the tendon housing 28 may not need to be expanded but may be configured to allow the tendon or other soft tissue 50 to fit within the tendon housing 28 without deflecting the arms 24, 26.

FIGS. 3A to 3D show various views of a tap plunger deployment system 60 connected to the single component anchor 20 of FIGS. 1A to 1C for engaging the tendon or other soft tissue and for positioning and deployment into a bone tunnel or channel. The anchor 20 may incorporate a screw fitting on the opposite surface of the tendon housing 28 from the surface that engages the tendon or other soft tissue 50. The deployment system 60 may incorporate a set screw that can be rotated into the anchor screw fitting to further attach the deployment system 60 to the anchor 20. Once the anchor 20 and attached tendon or other soft tissue 50 are positioned and secured within the bone tunnel or channel, the deployment plunger 60 is reverse rotated to disengage the anchor 20 and leave the attached tendon or other soft tissue secured within the bone tunnel or channel.

FIGS. 4A to 4C show various views of the anchor 20 of 3A to 3C engaged with a tendon or other soft tissue 50. The tendon 50 is partially or completely punctured with the central tissue penetrating member 30 of the anchor 20. The deployment plunger 60 is engaged to the anchor 20 and is used to manipulate the anchor 20 into engagement with the tendon or other soft tissue 50 and repositioning of the anchor/tendon combination into the bone tunnel or channel for deployment (see e.g. FIG. 5C).

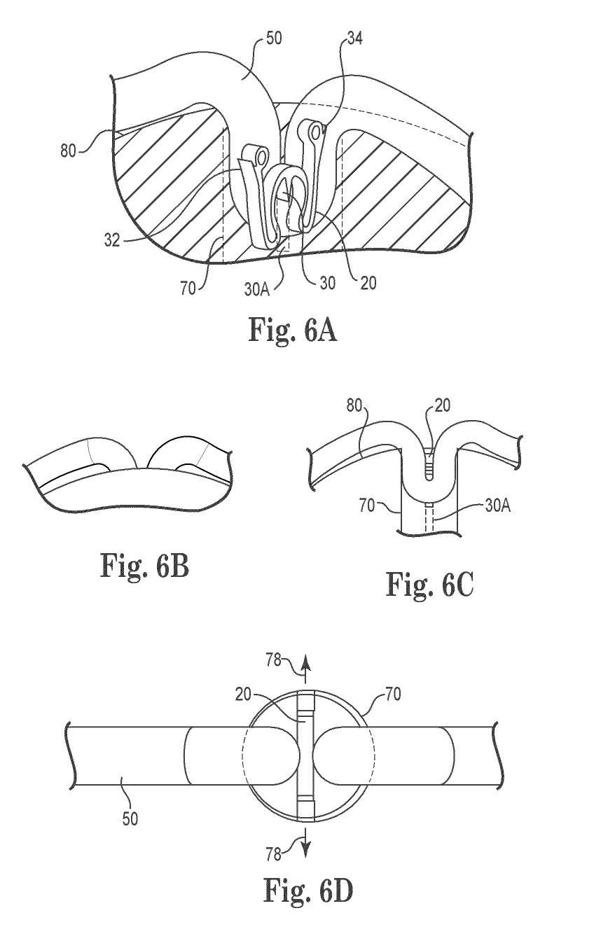

FIGS. 5A to 5D are various views of the anchor 20 engaged with tendon or other soft tissue 50 deployed into a bone tunnel or channel 70. FIGS. 6A to 6D are various views of the anchor 20 with the deployment system removed.

As the deployment plunger 60 is used to position the anchor 20 and the tendon 50 into the bone tunnel 70, the arms 24, 26 of the anchor 20 are allowed to deflect inward as the anchor is inserted through the opening 74 to the bone tunnel or channel 70.

Alternatively, the deployment system 60 may incorporate an outer sheath 76, pull rods, or other mechanism that compresses the arms 24, 26 into a lower profile for placement through the opening 74 to the bone tunnel or channel 70. Once positioned, the arms 24, 26 are allowed to expand in direction 78 into engagement with the bone 80 such that the lateral spikes 32, 34 partially penetrate into the cancellous bone 82 and the arms 24, 26 engage cortical bone 84 to ensure fixation (see e.g., FIG. 6A).

Multiple spikes may be incorporated along the arms 24, 26 of the anchor 20 to provide multiple engagement locations with the bone tunnel or channel 70 and better ensure engagement if the anchor 20 rotates within the tunnel or channel 70. Alternatively, a dilator or other expansion mechanisms may be introduced to manually expand the arms 24, 26 within the bone tunnel or channel 70 to further ensure engagement within bone tissue and attachment of the anchor 20 to the tendon or other soft tissue 50 within the bone tunnel or channel 70. The dilator can also be used to remove or reposition the anchor.

In an alternate embodiment, tissue penetrating member 30 may extend beyond the opening 42, so that when implanted, distal end 30A extends into the bone 80.

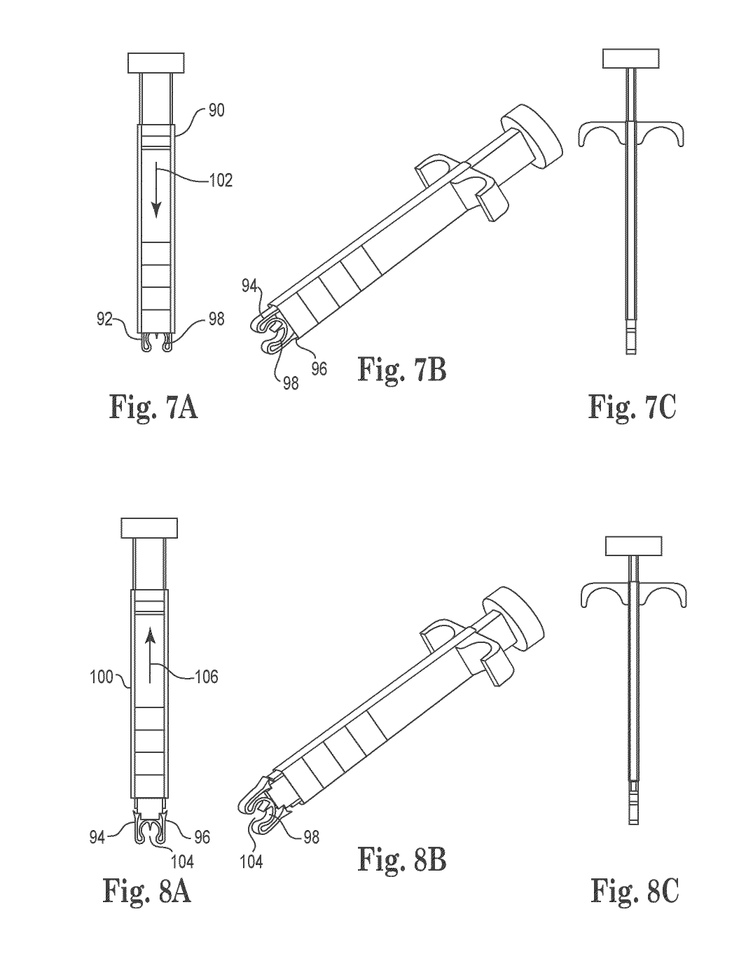

FIGS. 7A to 7C are various views of an alternative deployment system 90 for a single component anchor 92 that compresses the arms 94, 96 of the anchor 92 into a lower profile during deployment. This deployment system 90 also allows expanding the tendon housing 98 during engagement of the anchor 92 to the tendon or other soft tissue by compressing the arms 94, 96 of the anchor 92 while allowing the tendon housing 98 to expand into a larger opening. As the sheath 100 is extended in direction 102, the compression expands the tendon housing 98 outward to increase the size of the opening 104 for placement over and engagement to the tendon or other soft tissue. As the sheath is fully retracted in direction 106, as shown in FIGS. 8A to 8C, the tendon housing 98 is allowed to return to its smaller preformed shape engaging and compressing the tendon or other soft tissue. The arms 94, 96 are allowed to expand outward in order to secure with bone tissue in the tunnel or channel. The sheath 100 may be manipulated to provide partial compression of the arms 94, 96 for deployment into the bone tunnel or channel and allow full deployment once positioned with the engaged tendon.

The deployment sheath 100 in this embodiment is shown as having a rectangular cross-section. It should be noted that tubing having other cross-sections may be used (e.g. square, pentagonal, hexagonal, elliptical, circular, etc.) depending on the cross-sectional profile of the anchor 92. The deployment sheath 100 may compress the arms 94, 96 of the single component anchor 92, to provide column strength and rotational torque to enable manipulation of the anchor 92 into engagement with the tendon and placement of the combination into the bone tunnel or channel. The deployment sheath 100 may incorporate a distal opening and be configured to engage the arm eyelets (see e.g., items 36 and 38 in FIG. 1A) without fully encompassing the anchor 92 so the largest profile seen by the surgeon is not the deployment sheath 100 but is the anchor 92 ready for deployment.

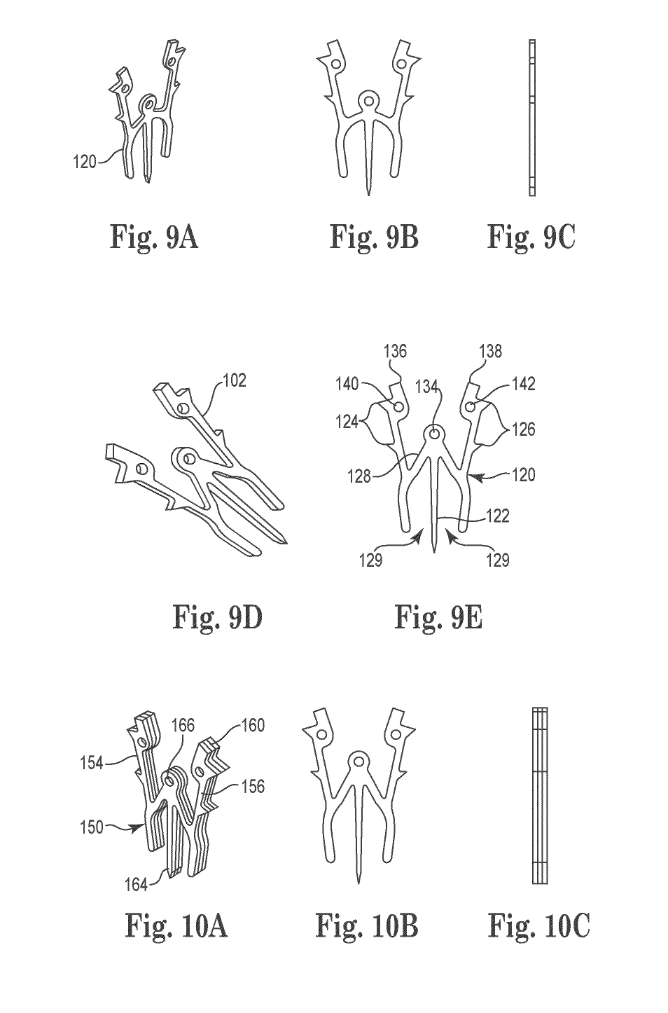

FIGS. 9A to 9E are various views of an alternative single component anchor 120 in accordance with the present disclosure in which the single component anchors are fabricated by bonding thin wafers 120 together into the stacked anchor 150, such as shown in FIGS. 10A to 10C.

The thin wafers 120 may be fabricated by chemical etching, laser cutting, water jet cutting, EDM, machining or other mechanism of a sheet of raw material into the desired anchor shape. Then individual wafers may be laser welded, ultrasonically welded, adhesively bonded, thermally bonded, spot welded, or soldered, depending on the type of material. Alternatively or additionally, rods may be inserted into the eyelets of the wafers and bonded to the wafers to form a single component anchor from multiple wafers bonded together.

These single component anchor 120 embodiments provide the same ability to engage a tendon or other soft tissue for positioning into a bone tunnel or channel and attachment of the anchor/tendon combination to bone tissue. The same features described above for the embodiment fabricated from a single piece of material 120 are incorporated in this stacked anchor 150, including the central tissue penetrating member 122 to engage and penetrate into tendon or other soft tissue, lateral arm spikes 124, 126 to engage bone tissue, a tendon housing 128 that forms an opening 129 that engages around the tendon or other soft tissue, and arms 130, 132 to allow compression into a lower profile for deployment into the bone tunnel or channel and engagement to bone once positioned.

A screw fitting 134 may be incorporated in the opposite surface of the tendon housing 128 from the surface that engages the tendon or other soft tissue so a deployment plunger with a set screw component may be removably attached. A stay suture or other filamentous material may be threaded through 134 to retain the anchor in the deployment sheath 100 and for retrieval in the case of premature deployment. The arms 130, 132 in these alternative embodiments include arm protrusions 136, 138 to which a deployment mechanism may engage to compress into a smaller profile without having to cover the entire anchor 120. As discussed previously, eyelets 140, 142 may permit the same function or may be modified into a protrusion that facilitates engagement with a deployment system capable of compressing the arms for deployment.

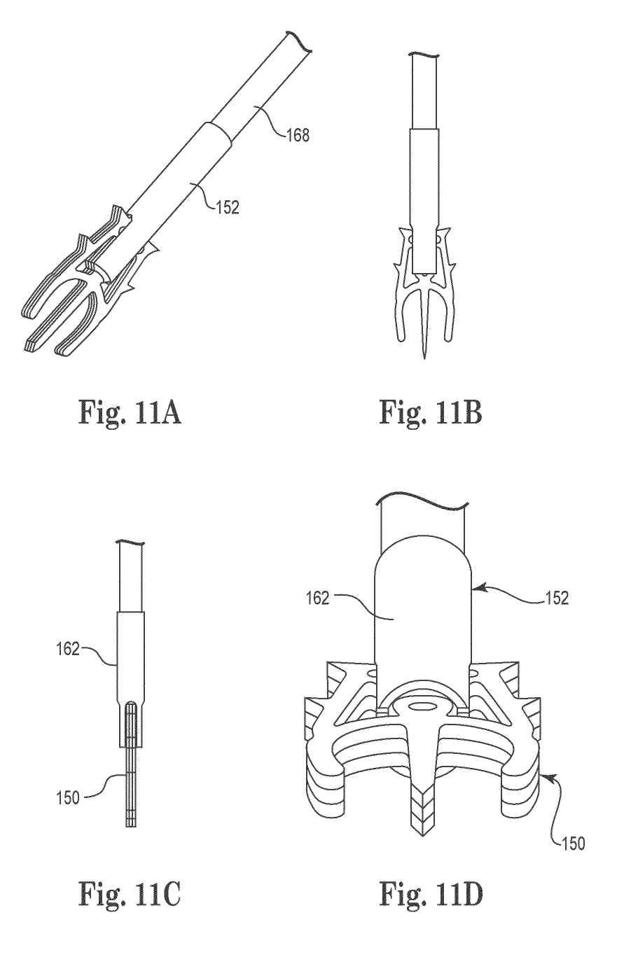

FIGS. 11A to 11D are various views of the stacked single component anchor 150 of FIGS. 10A to 10C connected to a tap deployment plunger 152 that compresses the arms 154, 156 of the anchor 150 by engaging the arm protrusions 158, 160 for deployment. This deployment plunger 152 further engages the anchor screw fitting 166 with a set screw and allows disengagement by reverse rotation of the plunger 152 relative to the deployed and secured anchor 150.

As illustrated the anchor 150 profile extends beyond the plunger 152 to allow the surgeon to fully visualize the anchor 150 both before and during deployment. The outer sheath 162 of the deployment plunger 152 allows retraction to allow the arms 154, 156 of the anchor 150 to return towards the preformed shape once positioning within the bone tunnel or channel to engage bone tissue and secure the anchor and tendon combination in place.

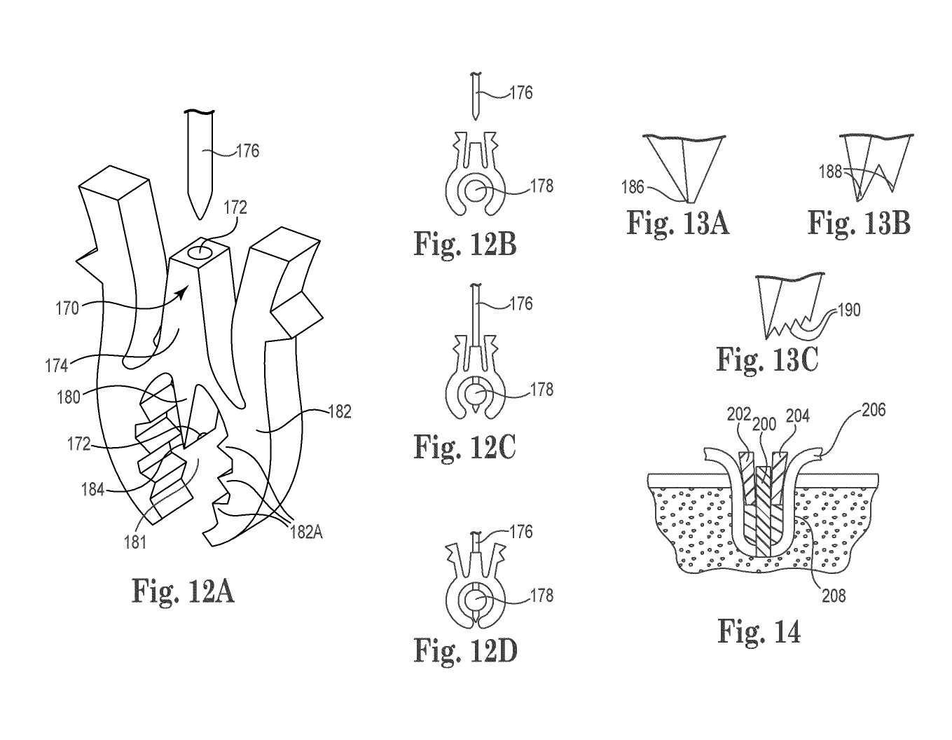

FIGS. 12A-12D illustrate an alternate anchor 170 in accordance with an embodiment of the present disclosure. Hole 172 extending through center portion 174 is sized to receive pin 176. In use, the anchor 170 is engaged with the tendon or soft tissue 178 as discussed above. As illustrated schematically in FIG. 12C, once in position, pin 176 is inserted through the hole 172 and preferably punctures or penetrates the tendon or soft tissue 178 and also, possibly the bone tissue. Excess portion of the pin 176 is then removed from the assembly, as illustrated in FIG. 12D. The pin 176 works in conjunction with central tissue penetrating member 180 to minimize slippage of the tendon within the tissue receiving region 181 of the tendon housing 182. In the illustrated embodiment, the tendon housing 182 optionally includes one or more serrations or barbs 182A oriented toward the tissue receiving region 181 to further engage the tendon.

In one embodiment, distal end 184 of the central tissue penetrating member 180 can be structured to enhance engagement with the tendon or soft tissue 178.

FIG. 13A illustrates a single point 186 embodiment, FIG. 13B illustrates a two-point 188 embodiment, and FIG. 13C illustrates a serrated 190 embodiment.

FIG. 14 illustrate an alternate embodiment of the present anchor 200 that includes secondary components 202, 204 added to engage and compress tendon 206 against bone tunnel 208.

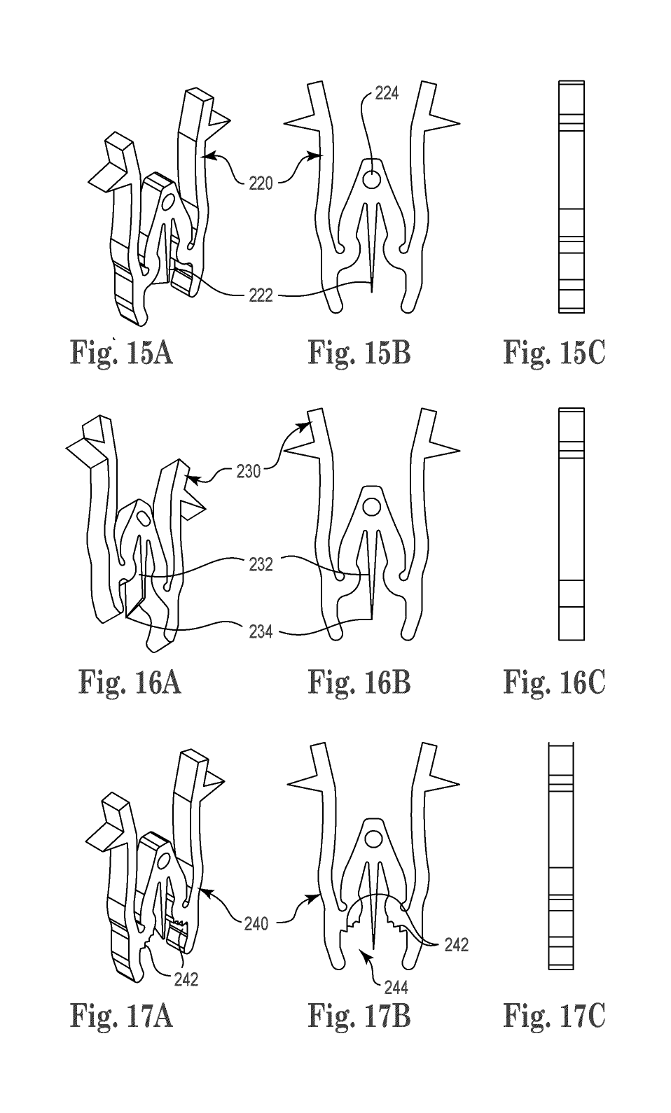

FIGS. 15A-15C illustrate an anchor 220 with a triangular prismatic central tissue penetrating member 222 in accordance with an embodiment of the present disclosure. Eyelet 224 can be used to attach suture material to the anchor 220, either to secure the tissue to the anchor 220, to secure the anchor 220 to the deployment tool, and/or to aid in removing the anchor 220. See e.g., FIG. 23

FIGS. 16A-16C illustrate an anchor 230 with a blade-like central tissue penetrating member 232 with an angled sharpened tip 234 in accordance with an embodiment of the present disclosure. FIGS. 17A-17C illustrate an anchor 240 with serrated gripping mechanisms 242 in the tissue engaging region 244 in accordance with an embodiment of the present disclosure.

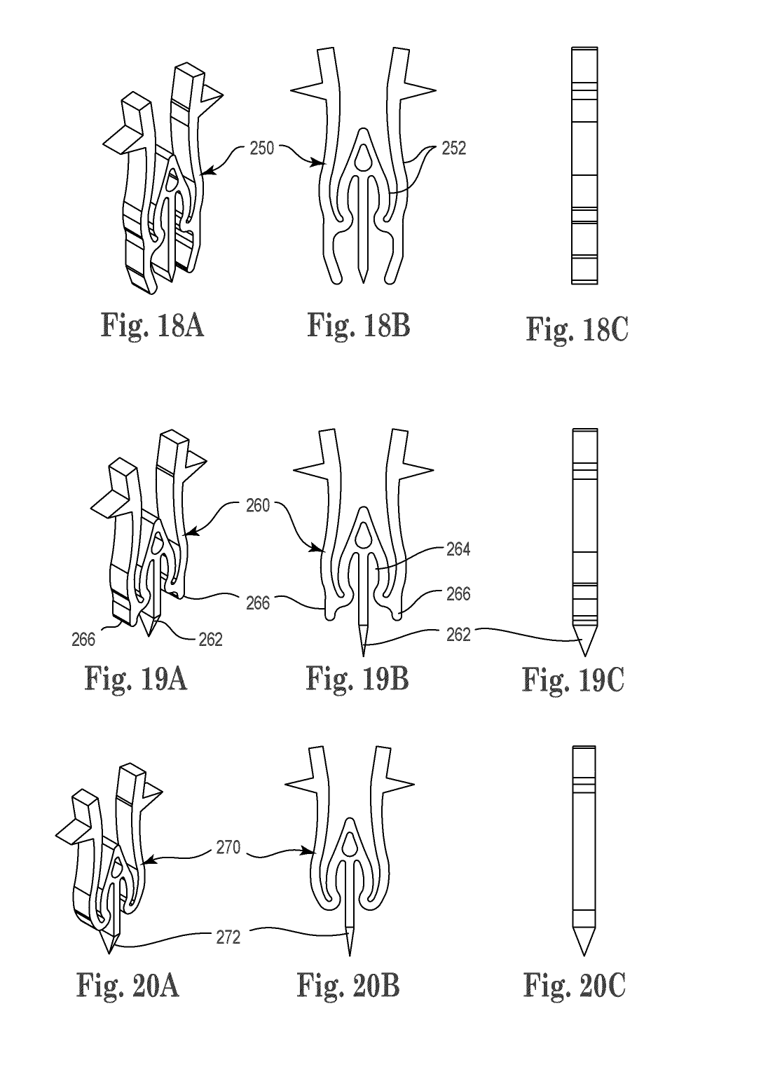

FIGS. 18A-18C illustrate an alternate anchor 250 with thinner walls 252 in accordance with an embodiment of the present disclosure. FIGS. 19A-19C illustrate an anchor 260 with an elongated central tissue penetrating member 262 that extends beyond the tissue engaging region 264 in accordance with an embodiment of the present disclosure. The central tissue penetrating member 262 is designed to optionally extend into the bone (see e.g., FIG. 6C). Central tissue penetrating member 262 controls the tissue, while protrusions 266 stabilize the tissue during movement of the tissue into the bone channel.

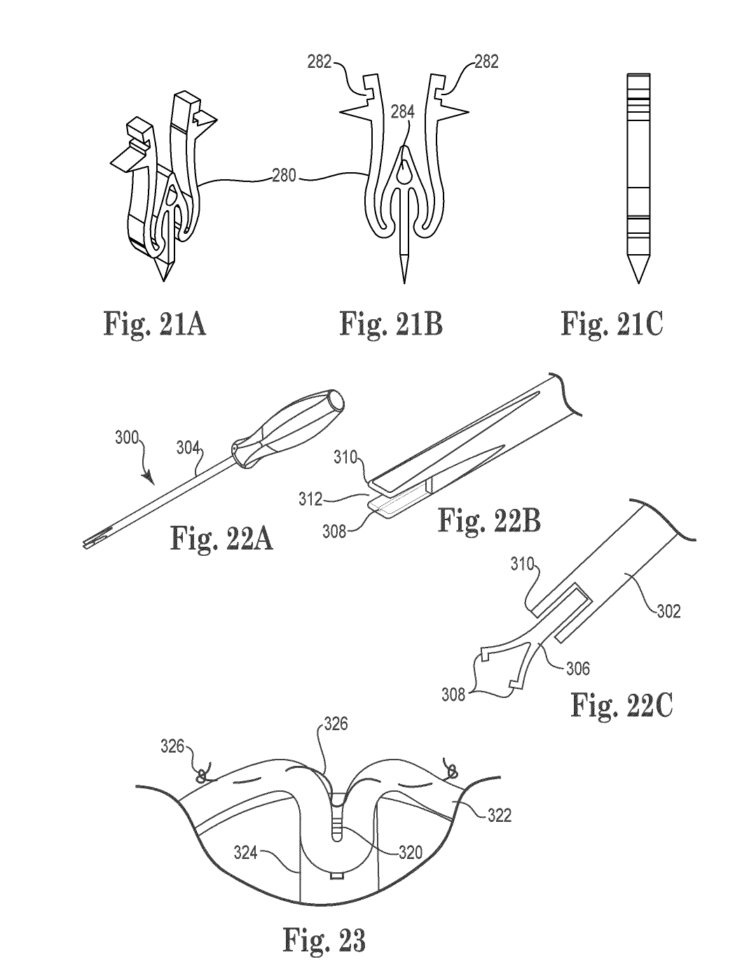

FIGS. 20A-20C illustrate an alternate anchor 270 with an elongated central tissue penetrating member 272 in accordance with an embodiment of the present disclosure. FIGS. 21A-21C illustrate an anchor 280 with notched arms 282 that engage with a retrieving device in accordance with an embodiment of the present disclosure. Hole 284 is provided to attach suture material. The hole 284 is preferably formed with rounded edges to minimize damage to the suture material.

FIG. 22A-22C illustrate various views of a deployment/retrieving device 300 in accordance with an embodiment of the present disclosure. Outer sleeve 302 slides along the tool body 304 to expose grasping portion 306. In the illustrated embodiment, protrusions 308 are configured to engage with notches 282 in the anchor 280 of FIGS. 21A-21C during deployment, repositioning, and/or removal.

Distal end 310 of the outer sleeve is preferably wider than the protrusions 308 in order to secure the tissue within space 312. The distal end 310 serves to hold the tissue to the device 300 during manipulation of the anchor 280 and insertion into the bone channel.

FIG. 23 is a side sectional view of anchor 320 securing tendon 322 to bone channel 324. Suture material 326 extending through an eyelet (see e.g., FIG. 19B) is attached to tendon 322 to enhance attachment to the anchor 320.

### Surgical Techniques

To accomplish biceps tenodesis, rotator cuff, other tendon, or other soft tissue fixation using the methods and devices described herein, standard surgical preparation of the site and/or arthroscopic portals for access of the region are performed. The joint is dilated with arthroscopic fluid if the procedure is to be performed arthroscopically. With open procedures, the device can easily be manipulated and deployed with a single hand. For arthroscopic procedures, the medial row fixation system is introduced through a standard 6 to 12 mm cannula placed into the joint.

The present tissue fixation system can be used with a variety of techniques. The specific details of the technique will vary depending on the anatomic structure being repaired and the device embodiments of the disclosure. Examples of specific uses will be described to demonstrate the versatility of the implant embodiments. The techniques relate to classes of procedures rather than individual procedures. They can be generally described as:

### Biceps Tenodesis

Create standard arthroscopic portals in which diagnostic arthroscopy is performed. This includes a posterior “soft spot” portal and lateral portal in addition to an anterior portal. Once the decision to perform a biceps tenodesis is made, the location of the tenodesis must be addressed. Whether intra-articular, in the bicipital groove, or sub pectoral, the tenodesis anchor may be used arthroscopically or in an open fashion.

Locate the desired position in the bicipital groove for reattachment of the biceps tendon. Using a spinal needle for anatomic location create a “biceps” portal just superficial to the desired position for tenodesis. Using a probe, or the cannula itself, pull or sweep aside the biceps tendon and drill a tunnel to a depth between about 20 millimeters (“mm”) to about 30 mm. Upon removal of the drill, allow the biceps tendon to return to its natural position lying directly over the tunnel.

Insert the tenodesis anchor through the cannulae and pass it over the biceps tendon. The central tissue penetrating member will pierce the tendon and control it. Cut the tendon with arthroscopic scissors or a biter to release it. Debride the tendon proximally from the superior labrum. Advance the tenodesis anchor into the tunnel with successive taps from a mallet. Test the fixation with a probe.

### Other Potential Uses of the Medial Row Anchor System

It should be appreciated that the medial row fixation system can be used for other indications involving the fixation of tendons, or other soft tissue to bone. The embodiments of this disclosure can be tailored to human anatomy, however, in some instances it may be possible for these to be tailored for use in other species such as horses, dogs, sheep, and pigs as well as invertebrates.

The size and scope of the disclosure provides additional advantages that include; providing an arthroscopic approach for the fixation of biceps tendon, other tendon, or other soft tissue to bone; reduction in the visible scars associated with open surgical procedures by using small port access allowed by the deployment device; reducing the complexity associated with arthroscopic knot tying, increasing the reliability of soft tissue attachment, and reducing the required surgical time as well as the level of complexity associated with these procedures.

The use of these devices can be applied to virtually all orthopedic procedures requiring fixation of tendon, or other soft tissue, into bone. The device will be useful for procedures whether performed with open dissection or with arthroscopic techniques. These include, but are not limited to:

Shoulder—
Long head of biceps tenodesis
Rotator cuff repair

Elbow—
Distal biceps tendon repairs
Medial (ulnar) collateral ligament reconstruction, The “Tommy John Procedure”
Lateral ulnar collateral ligament reconstruction—for Posterolateral rotatory instability of the elbow

Wrist—
Carpal Instability—Scapholunate and lunotriquetral ligament reconstructions, Blatt Capsulodesis
Thumb carpometacarpal arthroplasty (ligament reconstruction with tendon interposition—LRTI)

Hand—
Chronic thumb ulnar collateral ligament reconstruction (Gamekeeper's thumb)
Chronic thumb radial collateral ligament reconstruction
Finger metacarpophalangeal ligament reconstruction

Knee—
Medial collateral ligament repair/reconstructions with autograft or allograft
Lateral collateral ligament repair/reconstruction with autograft or allograft
Posterolateral reconstruction with autograft or allograft

Ankle and Foot—
Various lateral collateral ligament reconstructions (Watson-Jones/Chrisman Snook)

### Device Materials

Anchor and deployment instrument components can incorporate elastic properties or be plastically deformable. As such the anchor or deployment instrument components can be fabricated from various materials, including shape memory alloys, such as for example nickel titanium (e.g., Nitinol), shape memory polymers, polymers (i.e. PTFE, PEEK, polyurethane, urethane, silicone, polyimide, polypropylene, Polylactic Acid, Polyglycolic Acid, or other thermoset or thermoplastic, or elastomeric materials), and metal or alloys (i.e. titanium, CoCrMo, spring stainless steel, stainless steel 17-7, stainless steel 300 series, etc). Natural materials such as collagen may also be used.

In some embodiments the anchor components are resorbable. In other embodiments the anchor components will have limited or no resorption characteristics. The anchor components described in this patent can be made in part or solely of one material. Alternatively, the components of the anchors or deployment instruments can be composed of metal and/or polymer components fabricated into composite devices. For example, low surface area and thin metal or metal alloy components can be insert molded with a polymer (e.g. polypropylene) to produce a composite device. Some embodiments may include parts that are resorbable and some that are not.

Fabrication of these components can be performed using techniques familiar with manufacturing methods by people skilled in the art of metals, polymers, shape memory alloys, shape memory polymers, collagen, or composite materials. Sample techniques will include but are not limited to extrusion, casting, press-forging, rolling, injection molding, or pressing methods for the fabrication of parts for the above materials.

In specific instances, the use of techniques related to modification of polymer chemistry to adjust the shape memory characteristics related to thermal conditions and elastic properties of the polymer will be utilized. With respect to shape memory metal materials, it is possible to utilize the thermal characteristics of the specified composition to fabricate components with the geometry and features required for the device component. Proper thermal forming and quenching is required to process the material and is generally known to someone skilled in the art of using, processing, and fabricating components out of shape memory materials. In some embodiments several components may require parts using standard machining techniques typically known to someone skilled in the art of machining. For example, use of CNC, EDM, laser cutting, water jet cutting, polishing methods, and other machining techniques. Several embodiments may also require bonding or welding of components and include adhesives, laser welding, soldering, or other means of attachment.

Anchor components that include spikes or tabs can be fabricated from any stock materials typically known from someone well versed in the art of medical device manufacturing. Attachment of other components to these embodiments can be performed by tying, welding, bonding, clamping, embedding, or use of other such means. In some embodiments, these anchors can be mechanically polished or electropolished to produce smooth surfaces.

Various embodiments of the anchor components described can be coated with or encapsulated with a covering of a polymer material that can allow for the use of anti-proliferative, antibiotic, angiogenic, growth factors, anti-cancer, or other pharmacological substances that may provide a benefit related to inhibiting or promoting biological proliferation. These substances can be loaded into the encapsulating coatings and be allowed to elute into the surrounding matrix, tissues, or space that it sits. The time course of delivery can be tailored to the intended application by varying the polymer or the characteristics of the coating. Such coatings with pharmacological substances can act as anti-proliferative treatments or can aid in the healing response of the tissue being treated. Furthermore, these coatings can act to reduce the local coagulation or hyperplastic response near the anchor.

Where a range of values is provided, it is understood that each intervening value, to the tenth of the unit of the lower limit unless the context clearly dictates otherwise, between the upper and lower limit of that range and any other stated or intervening value in that stated range is encompassed within the embodiments. The upper and lower limits of these smaller ranges which may independently be included in the smaller ranges is also encompassed within the embodiments, subject to any specifically excluded limit in the stated range. Where the stated range includes one or both of the limits, ranges excluding either both of those included limits are also included in the embodiments.

Unless defined otherwise, all technical and scientific terms used herein have the same meaning as commonly understood by one of ordinary skill in the art to which these embodiments belong. Although any methods and materials similar or equivalent to those described herein can also be used in the practice or testing of the present embodiments, the preferred methods and materials are now described. All patents and publications mentioned herein, including those cited in the Background of the application, are hereby incorporated by reference to disclose and described the methods and/or materials in connection with which the publications are cited.

The publications discussed herein are provided solely for their disclosure prior to the filing date of the present application. Nothing herein is to be construed as an admission that the present embodiments are not entitled to antedate such publication by virtue of prior invention. Further, the dates of publication provided may be different from the actual publication dates which may need to be independently confirmed.

Other embodiments are possible. Although the description above contains much specificity, these should not be construed as limiting the scope of the embodiments, but as merely providing illustrations of some of the presently preferred embodiments. It is also contemplated that various combinations or sub-combinations of the specific features and aspects of the embodiments may be made and still fall within the scope of the disclosure. It should be understood that various features and aspects of the disclosed embodiments can be combined with or substituted for one another in order to form varying modes of the disclosed embodiments. Thus, it is intended that the scope of at least some of the present embodiments herein disclosed should not be limited by the particular disclosed embodiments described above.

Thus the scope of this disclosure should be determined by the appended claims and their legal equivalents. Therefore, it will be appreciated that the scope of the present disclosure fully encompasses other embodiments which may become obvious to those skilled in the art, and that the scope of the present embodiments is accordingly to be limited by nothing other than the appended claims, in which reference to an element in the singular is not intended to mean “one and only one” unless explicitly so stated, but rather “one or more.” All structural, chemical, and functional equivalents to the elements of the above-described preferred embodiment that are known to those of ordinary skill in the art are expressly incorporated herein by reference and are intended to be encompassed by the present claims. Moreover, it is not necessary for a device or method to address each and every problem sought to be solved by the present embodiments, for it to be encompassed by the present claims. Furthermore, no element, component, or method step in the present disclosure is intended to be dedicated to the public regardless of whether the element, component, or method step is explicitly recited in the claims.

What is claimed is:

1. A tissue fixation system for attaching tissue to a channel formed in a bone, the tissue fixation system comprising:
a generally planar, single component anchor comprising a partially enclosed tissue engaging region with an opening oriented in a distal direction, the tissue engaging region adapted to compressively engage the tissue, a pair of arms extending in a generally proximal direction with structures adapted to engage with the bone, such that displacement of the arms toward each other in a compressed configuration increases the size of the opening to facilitate engagement with the tissue, and the arms expanding outward in an expanded configuration after deployment to engage the bone; and
at least one tissue penetrating member engaged with the anchor and extending into at least the tissue engaging region.

2. The tissue fixation system of claim 1 wherein the tissue penetrating member in integrally formed with the structure.

3. The tissue fixation system of claim 1 comprising a hole extending through the anchor to the tissue engaging region, and the tissue penetrating member is a discrete component sized to slide through the hole and into tissue located in the tissue engaging region.

4. The tissue fixation system of claim 1 comprising one or more eyelets formed in one or more of the arms.

5. The tissue fixation system of claim 1 wherein the anchor includes one or more serrations or barbs oriented toward the tissue engaging region.

6. The tissue fixation system of claim 1 wherein the structures on the arms are configured to engage cortical or cancellous bone within the channel.

7. The tissue fixation system of claim 1 comprising a deployment system adapted to engage proximal ends of the arms.

8. The tissue fixation system of claim 1 comprising a deployment system adapted to engage proximal ends of the arms in a compressed configuration, and to release the arms when the tissue fixation system is in the channel.

9. The tissue fixation system of claim 8 wherein the deployment system comprises a sheath that slidingly engages proximal ends of the arms in the compress configuration.

10. The tissue fixation system of claim 1 comprising a plurality of generally planar, single component anchors laminated to form a unitary structure.

11. The tissue fixation system of claim 1 wherein a distal end of the tissue penetrating member comprises one or more of points, blades, teeth, or serrations.

12. The tissue fixation system of claim 1 comprising one or more secondary components adapted to be inserted into the channel with the anchor.

13. A method of attaching tissue to a channel formed in a bone, the method comprising the steps of:
compressing proximally extending arms on a generally planar, single component anchor to increase the size of a distally oriented opening to a tissue engaging region;
engaging the tissue engaging region with the tissue;
engaging at least one tissue penetrating member extending into the tissue engaging region with the tissue;
inserting the anchor and the tissue into the channel formed in the bone; and
releasing the arms so that structures on the arms engage cortical or cancellous bone within the channel.

14. The method of claim 13 wherein the step of inserting the anchor into the channel is performed without rotation.

15. The method of claim 13 wherein tissue penetrating member engages with the tissue substantially simultaneously with the tissue engaging region.

16. The method of claim 13 comprising the step of sliding the tissue penetrating member into a hole extending through the anchor and into at least the tissue engaging region.

17. The method of claim 13 comprising engaging proximal ends of the arms with a deployment system.

18. The method of claim 17 comprising the steps of:
retaining the arms in a compressed configuration using the deployment system; and
releasing the arms when the anchor is in the channel.

19. The method of claim 13 comprising laminating a plurality of generally planar, single component anchor laminated to form a unitary structure.

20. The method of claim 13 comprising inserting one or more secondary components into the channel with the anchor.

21. The method of claim 13 comprising the steps of:
engaging a tool with the proximal extending arms;
compressing the proximally extending arms to release the structures on the arms from the cortical or cancellous bone within the channel; and
removing the anchor from the channel.
